(19) United States
(12) Patent Application Publication
(10) Pub. No.: US 2022/0408862 A1
(43) Pub. Date: Dec. 29, 2022

(54) Mask for Detecting Virus of Respiratory Tract Infection

(71) Applicant: KKT Holdings Syndicate, Dover, DE (US)

(72) Inventor: Tzu-Yi KUO, Taipei City (TW)

(21) Appl. No.: 17/849,572

(22) Filed: Jun. 24, 2022

Related U.S. Application Data
(60) Provisional application No. 63/214,452, filed on Jun. 24, 2021.

(51) Int. Cl. A41D 13/11; A62B 23/02; A41D 31/102; A41D 31/30

(52) U.S. Cl. CPC A41D 13/1107; A62B 23/025; A41D 31/102; A41D 31/305

(57) ABSTRACT

This invention provides a respirator for detecting viruses which cause respiratory tract infection. The respirator comprises an outer layer, an inner layer, a rapid screening reagent between the outer layer and the inner layer, and a water-resist membrane between the rapid screening reagent and the inner layer. A sample pad of the rapid screening reagent is located on a region close to the nostrils, such that air from the nostrils will arrive the sample pad. Viruses inside the air with vapors or moistures will flow inside the rapid screening reagent through a conjugate pad, a nitrocellulose membrane, and an absorption pad. At least one test line will indicate a corresponding virus.

### FIELD OF THE INVENTION

The invention relates to a wearable rapid screening reagent, and more particularly to a mask or respirator for detecting viruses which cause respiratory tract infection.

### BACKGROUND OF THE INVENTION

Respiratory tract infections are infectious diseases involving the respiratory tract. An infection of this type usually is further classified as an upper respiratory tract infection (URI or URTI) or a lower respiratory tract infection (LRI or LRTI). Lower respiratory infections, such as pneumonia, are generally more severe than upper respiratory infections, such as the common cold.

The upper respiratory tract is considered the airway above the glottis or vocal cords; sometimes, it is taken as the tract above the cricoid cartilage. This part of the tract includes the nose, sinuses, pharynx, and larynx. Typical infections of the upper respiratory tract include tonsillitis, pharyngitis, laryngitis, sinusitis, otitis media, certain influenza types, and the common cold. Symptoms of URIs can include cough, sore throat, runny nose, nasal congestion, headache, low-grade fever, facial pressure, and sneezing.

The lower respiratory tract consists of the trachea (windpipe), bronchial tubes, bronchioles, and the lungs. The two most common LRIs are bronchitis and pneumonia. Influenza affects both the upper and lower respiratory tracts, but more dangerous strains such as the highly pernicious H5N1 tend to bind to receptors deep in the lungs.

Causes of the respiratory tract infection includes bacteria, virus, fungi, and parasites, wherein virus is the most difficult one to be detected. For symptomatic people infected by the COVID 19 are diagnosed by using PCR, however the asymptomatic people, who can also transmit the viruses, can't be confirmed if there is no routine screening. For the health-care or medical personnel in the nursing home or hospital, they face large amounts of patience and will not be confirmed infected unless the symptoms appear. However, viruses have been transmitted in the hospital or nursing home before.

Thus, an invention is necessary to solve the issues mentioned above.

### BRIEF SUMMARY OF THE INVENTION

The object of this invention is to provide a mask or respirator for detecting viruses which may cause respiratory tract infection. Thus, the medical personnel in the nursing home or hospital can test oneself if infected while the mask or respirator is taken off.

This invention also provides a wearable rapid screening reagent such that people can test themselves at any place and at any time.

This invention also provides a rapid screening reagent which can detect or test more than one kind of viruses causing respiratory tract infection.

Accordingly, the invention provides a respirator, which comprises an outer layer and a transparent inner layer; a screening reagent, between the outer layer and the inner layer, being fastened to the inner layer, wherein the inner layer includes a region for a sample pad of the screening reagent, such that air from nostrils targets to the region; and a water-resist membrane, between the screening reagent and the inner layer, covering the screening reagent except the region.

In one embodiment, the present invention may further comprise a filter layer between said outer layer and said screening reagent, and a support layer between said filter layer and said screening reagent.

In one embodiment of the present invention, the outer layer is water repellent non-woven fabric, the filter layer is melt-blown non-woven fabric, the support layer is modacrylic, and the inner layer is skin-friendly non-woven fabric.

In one embodiment of the present invention, the outer layer and inner layer are polypropylene.

In one embodiment of the present invention, the screening reagent comprises the sample pad for receiving the sample; a conjugate pad including a first reagent for binding a first target molecule in the sample; a nitrocellulose membrane including a first test line with a first antibody; an absorption pad for making the sample flow through said conjugate pad and said nitrocellulose membrane by using capillary flow, thereby the first test line showing a first signal while the first reagent moving through the first test line; and a soft adhesive board for fastening said sample pad, said conjugate pad, said nitrocellulose membrane, and said absorption pad.

In one embodiment of the present invention, the nitrocellulose membrane including a control line for testing the sample passing to the absorption pad.

In one embodiment of the present invention, the conjugate pad including a second reagent for binding a second target molecule in the sample, and the nitrocellulose membrane including a second test line with a second antibody.

In one embodiment of the present invention, the conjugate pad including a third reagent for binding a third target molecule in the sample, and the nitrocellulose membrane including a third test line with a third antibody.

In one embodiment of the present invention, the sample comprises a virus causing respiratory tract infection.

In one embodiment of the present invention, the virus is selected from the group consisting of rhinovirus, coronavirus, influenza virus, respiratory syncytial virus, adenovirus, and parainfluenza.

In one embodiment of the present invention, the coronavirus is selected from the group consisting of HCoV-229E, HCoV-OC43, HCoV-NL63, HCoV-HKU1, SARS-CoV, MERS-CoV, and SARS-CoV-2.

The present invention also provides a wearable rapid screening reagent, which comprises a mask including an outer layer and an inner layer, wherein the rapid screening reagent, between said outer layer and said inner layer, fastens to said inner layer, wherein said inner layer includes a region for a sample pad of the rapid screening reagent, such that gas from nostrils targets to the region; and a water-resist membrane, between the rapid screening reagent and said inner layer, covering the rapid screening reagent except the region.

In one embodiment of the present invention, the mask comprises a filter layer between said outer layer and said screening reagent, and a support layer between said filter layer and said screening reagent.

In one embodiment of the present invention, the outer layer is water repellent non-woven fabric, said filter layer is melt-blown non-woven fabric, said support layer is modacrylic, and said inner layer is skin-friendly non-woven fabric.

In one embodiment of the present invention, the screening reagent comprises said sample pad for receiving the sample; a conjugate pad including a first reagent for binding a first target molecule in the sample; a nitrocellulose membrane including a first test line with a first antibody; an absorption pad for making the sample flow through said conjugate pad and said nitrocellulose membrane by using capillary flow, thereby the first test line showing a first signal while the first reagent moving through the first test line; and a soft adhesive board for fastening said sample pad, said conjugate pad, said nitrocellulose membrane, and said absorption pad.

In one embodiment of the present invention, the nitrocellulose membrane including a control line for testing the sample passing to the absorption pad.

In one embodiment of the present invention, the conjugate pad including a second reagent for binding a second target molecule in the sample, and the nitrocellulose membrane including a second test line with a second antibody.

In one embodiment of the present invention, the conjugate pad including a third reagent for binding a third target molecule in the sample, and the nitrocellulose membrane including a third test line with a third antibody.

In one embodiment of the present invention, the sample comprises a virus causing respiratory tract infection.

In one embodiment of the present invention, the virus is selected from the group consisting of rhinovirus, coronavirus, influenza virus, respiratory syncytial virus, adenovirus, and parainfluenza.

In one embodiment of the present invention, the coronavirus is selected from the group consisting of HCoV-229E, HCoV-OC43, HCoV-NL63, HCoV-HKU1, SARS-CoV, MERS-CoV, and SARS-CoV-2.

The present invention further provides a method for detecting virus, which comprises steps of: providing a mask including an outer layer and an inner layer; providing a screening reagent, between said outer layer and said inner layer, fastens to said inner layer, wherein said inner layer includes a region for a sample pad of the rapid screening reagent, such that gas from nostrils targets to the region; providing a water-resist membrane, between the rapid screening reagent and said inner layer, covering the rapid screening reagent except the region; wearing the mask; checking if a signal of a test line on a nitrocellulose membrane of the screening reagent is shown.

Other advantages of the present invention will become apparent from the following description taken in conjunction with the accompanying drawings wherein are set forth, by way of illustration and example, certain embodiments of the present invention.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a schematic side-view illustration of a rapid screening reagent in according to one embodiment of the present invention;

FIG. 2 is a schematic top-view illustration of a rapid screening reagent in according to one embodiment of the present invention;

FIG. 3 is a schematic exploded-view illustration of a mask in according to embodiments of the present invention;

FIG. 4 is a schematic bottom-view illustration of a mask in accordance with several embodiments of the present invention;

FIG. 5 is a schematic cross-sectional illustration of a wearable rapid screening reagent in accordance with one embodiment of the present invention;

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE INVENTION

Various example embodiments of the present invention will now be described more fully with reference to the accompanying drawings in which some example embodiments of the invention are shown. Without limiting the scope of the protection of the present invention, all the description and drawings of the embodiments will exemplarily be referred to rapid screening reagent and mask or respirator. However, the embodiments are not be used to limit the present invention to rapid screening reagent and mask or respirator.

As used herein, the term “mask” or “respirator” generally refers to an article which can cover nose and mouth to prevent the inhalation of noxious substances, pathogens, or the like.

As used herein, the term “rapid screening reagent” or “screening reagent” generally refers to a device which can test if there is virus from a sample. The term “rapid” in the present invention refers to a test time about several minutes to hours, which is faster than PCR.

Accordingly, while example embodiments of the invention are capable of various modifications and alternative forms, embodiments thereof are shown by way of example in the drawings and will herein be described in detail. It should be understood, however, that there is no intent to limit example embodiments of the invention to the particular forms disclosed, but on the contrary, example embodiments of the invention are to cover all modifications, equivalents, and alternatives falling within the scope of the invention.

Pneumonia is an inflammatory condition of the lung primarily affecting the small air sacs known as alveoli. Symptoms typically include some combination of productive or dry cough, chest pain, fever and difficulty breathing. The severity of the condition is variable. Pneumonia is usually caused by infection with viruses or bacteria, and less commonly by other microorganisms. Identifying the responsible pathogen can be difficult. Diagnosis is often based on symptoms and physical examination. Chest X-rays, blood tests, and culture of the sputum may help confirm the diagnosis. The disease may be classified by where it was acquired, such as community- or hospital-acquired or healthcare-associated pneumonia. Risk factors for pneumonia include cystic fibrosis, chronic obstructive pulmonary disease (COPD), sickle cell disease, asthma, diabetes, heart failure, a history of smoking, a poor ability to cough (such as following a stroke), and a weak immune system.

In adults, viruses account for about one third of pneumonia cases, and in children for about 15% of them. Commonly implicated agents include rhinoviruses, coronaviruses, influenza virus, respiratory syncytial virus (RSV), adenovirus, and parainfluenza. Herpes simplex virus rarely causes pneumonia, except in groups such as newborns, persons with cancer, transplant recipients, and people with significant burns. After organ transplantation or in otherwise immunocompromised persons, there are high rates of cytomegalovirus pneumonia. Those with viral infections may be secondarily infected with the bacteria Streptococcus pneumoniae, Staphylococcus aureus, or Haemophilus influenzae, particularly when other health problems are present. Different viruses predominate at different times of the year; during flu season, for example, influenza may account for more than half of all viral cases. Outbreaks of other viruses also occur occasionally, including hantaviruses and coronaviruses. Severe acute respiratory syndrome coronavirus 2 (SARS-CoV-2) can also result in pneumonia.

Coronaviruses are a group of envelop viruses which lead to diseases in birds and mammals as well as human. Seven coronaviruses have been discovered in humans that can cause mild to lethal respiratory tract infections. HCoV-229E, HCoV-OC43, HCoV-NL63, and HCoV-HKU1 are the low-risk members of this family and the reason for some common colds. Besides, SARS-CoV, MERS-CoV, and newly identified SARS-CoV-2, which is also known as 2019-nCoV, are the more dangerous viruses. Due to the rapid spread of this novel coronavirus and its related disease, COVID-19, a reliable, simple, fast, and low-cost detection method is necessary for patient diagnosis and tracking worldwide.

Coronaviruses are members of the Coronaviridae family, which belongs to the

Nidovirales order. These viruses are enveloped, non-segmented, positive-sense, and single-stranded RNA viruses that cause mild or severe diseases in some birds and mammals, including humans. Their genome size is about 30 kilobases (kb) which consist of non-structural open reading frames (ORFs) near the 5′-end and at least four structural proteins near the 3′-end, including membrane (M), envelope (E), spike (S), and nucleocapsid (N) proteins. The coronavirus name derives from their solar corona appearance, which is formed due to the club-shaped spikes that project from the surface of the virion.

The first study on coronaviruses was reported in 1931, while the first human coronaviruses were identified in the 1960s. HCoV-229E and HCoV-OC43 were discovered as responsible viruses for some cases of cold and respiratory tract infection. According to their genome structure, HCoV-229E is classified in the alpha-coronaviruses (or group 1) subgroup while HCoV-OC43 belongs to beta-coronaviruses (or group 2) subgroup.

Severe acute respiratory syndrome (SARS) was reported for the first time in November 2002 in China, and SARS-associated coronavirus (SARS-CoV) was identified in March 2003 as the third human coronavirus. This dangerous virus was characterized and classified in the beta-coronaviruses subgroup.

After the SARS epidemic, several research investigations have been done to find the new human coronaviruses. HCoV-NL63 was reported in 2004 in the Netherlands as a member of alpha-coronaviruses. Afterward, the next human beta-coronavirus was identified in 2005, which was named HCoV-HKU1. In a few years after the discovery of HCoV-NL63 and HCoV-HKU1, numerous reports were published about the presence of these coronaviruses in The sixth human coronavirus was emerged in the Middle East in 2012 and named Middle East respiratory syndrome-CoV (MERS-CoV). MERS-CoV is another member of the beta-coronaviruses subgroup and could infect humans and dromedary camels. So, dromedary camels are known as the possible zoonotic source for MERS-CoV. Furthermore, MERS was a severe lower respiratory tract infection with a high fatality rate similar to SARS.

The last human coronavirus appeared in December 2019 in Wuhan, China, which was named 2019 novel coronavirus (2019-nCoV) by the World Health Organization (WHO) and then renamed to severe acute respiratory syndrome coronavirus 2 (SARS-CoV-2), due to its similarity to SARS-CoV. Also, WHO named the related disease as coronavirus disease 2019 (COVID-19). The ongoing pandemic is growing fast, and between 100,000 and 300,000 new cases per day were reported over the past month. Furthermore, about sixteen million laboratory-confirmed cases around the world and more than 600,000 deaths have been reported until 25 Jul. 2020.

COVID-19 testing involves analyzing samples to assess the current or past presence of SARS-CoV-2. The two main branches detect either the presence of the virus or of antibodies produced in response to infection. Molecular tests for viral presence through its molecular components are used to diagnose individual cases and to allow public health authorities to trace and contain outbreaks. Antibody tests (serology immunoassays) instead show whether someone once had the disease. They are less useful for diagnosing current infections because antibodies may not develop for weeks after infection. It is used to assess disease prevalence, which aids the estimation of the infection fatality rate.

Individual jurisdictions have adopted varied testing protocols, including whom to test, how often to test, analysis protocols, sample collection and the uses of test results. This variation has likely significantly impacted reported statistics, including case and test numbers, case fatality rates and case demographics. Because SARS-CoV-2 transmission occurs days after exposure (and before onset of symptoms) there is an urgent need for frequent surveillance and rapid availability of results.

Positive viral tests indicate a current infection, while positive antibody tests indicate a prior infection. Other techniques include a CT (computerized tomography) scan, checking for elevated body temperature, checking for low blood oxygen level, and the deployment of detection dogs at airports.

Due to the rapid spread of COVID-19, a reliable detection method is needed for patient diagnosis and tracking worldwide, especially in the early stages of the disease, to slow and try to control the pandemic. WHO has recommended nucleic acid amplification tests (NAAT), such as reverse transcription polymerase chain reaction (RT-PCR), for this purpose.

Detection of the virus is usually done either by looking for the virus' inner DNA, or pieces of protein on the outside of the virus. Tests that look for the viral antigens (parts of the virus) are called antigen tests.

There are multiple types of tests that look for the virus by detecting the presence of the virus's DNA. These are called molecular tests, after molecular biology. As of 2021, the most common form of molecular test is the reverse transcription polymerase chain reaction (RT-PCR) test. Other methods used in molecular tests include CRISPR, isothermal nucleic acid amplification, digital polymerase chain reaction, microarray analysis, and next-generation sequencing.

Polymerase chain reaction (PCR) is a process that amplifies (replicates) a small, well-defined segment of DNA many hundreds of thousands of times, creating enough of it for analysis. Test samples are treated with certain chemicals that allow DNA to be extracted. Reverse transcription converts RNA into DNA.

Reverse transcription polymerase chain reaction (RT-PCR) first uses reverse transcription to obtain DNA, followed by PCR to amplify that DNA, creating enough to be analyzed. RT-PCR can thereby detect SARS-CoV-2, which contains only RNA. The RT-PCR process generally requires a few hours. These tests are also referred to as molecular or genetic assays.

Real-time PCR (qPCR) provides advantages including automation, higher-throughput and more reliable instrumentation. The combined technique has been described as real-time RT-PCR or quantitative RT-PCR and is sometimes abbreviated qRT-PCR, rRT-PCR or RT-qPCR, although sometimes RT-PCR or PCR are used.

Nevertheless, the RT-PCR method is a relatively expensive and time-consuming technique and needs an expert technician, as well as specialized equipment compared to other diagnostic methods. It has also shown some false-negative results (even more than 30%) in COVID-19 diagnosis compare to chest CT imaging technique results. Furthermore, except chest CT-imaging and host antibody detection (serological assays) that have been used for diagnosis purposes, there are other methods for SARS-CoV-2 direct detection, such as RT-LAMP, viral RNA biosensing, and viral protein biosensors.

Antigen tests may be one way to scale up testing to much greater levels. Isothermal nucleic acid amplification tests can process only one sample at a time per machine. RT-PCR tests are accurate but require too much time, energy and trained personnel to run the tests.

In the health-care house or hospital, medical personnel work in such a high-risk environment. Although some protection equipment, such as mask or respirator is provided, infected risk is still high. Hence, medical personnel should test themselves frequently if she or he is infected or asymptotic after a surgery or a medical process. In the present invention, because the medical personnel always wear a mask or respirator, it is possible to integrate a rapid screening reagent inside the mask or respirator. When the medical personnel take off the mask or respirator, she or he can determine if being infected in a medical process. When the mask or respirator is taken off, PCR test, or even chest CT scan and blood plasma test should be proceeded, if the rapid screening reagent indicates infection. In the present invention, the rapid screening is lateral flow test.

Lateral flow tests (LFTs) derive from paper chromatography, which was developed in 1943 by Martin and Synge, and elaborated in 1944 by Consden, Gordon and Martin. There was an explosion of activity in this field after 1945. Lateral flow tests (LFTs), also known as lateral flow immunochromatographic assays or rapid tests, are simple devices intended to detect the presence of a target substance in a liquid sample without the need for specialized and costly equipment. These tests are widely used in medical diagnostics for home testing, point of care testing, or laboratory use. For instance, the home pregnancy test is an LFT that detects a certain hormone. These tests are simple, economic and generally show results in around five to 30 minutes. Many lab-based applications increase the sensitivity of simple LFTs by employing additional dedicated equipment.

LFTs operate on the same principles as the enzyme-linked immunosorbent assays (ELISA). In essence, these tests run the liquid sample along the surface of a pad with reactive molecules that show a visual positive or negative result. The pads are based on a series of capillary beds, such as pieces of porous paper, microstructured polymer, or sintered polymer. Each of these pads has the capacity to transport fluid (e.g., urine, blood, saliva) spontaneously.

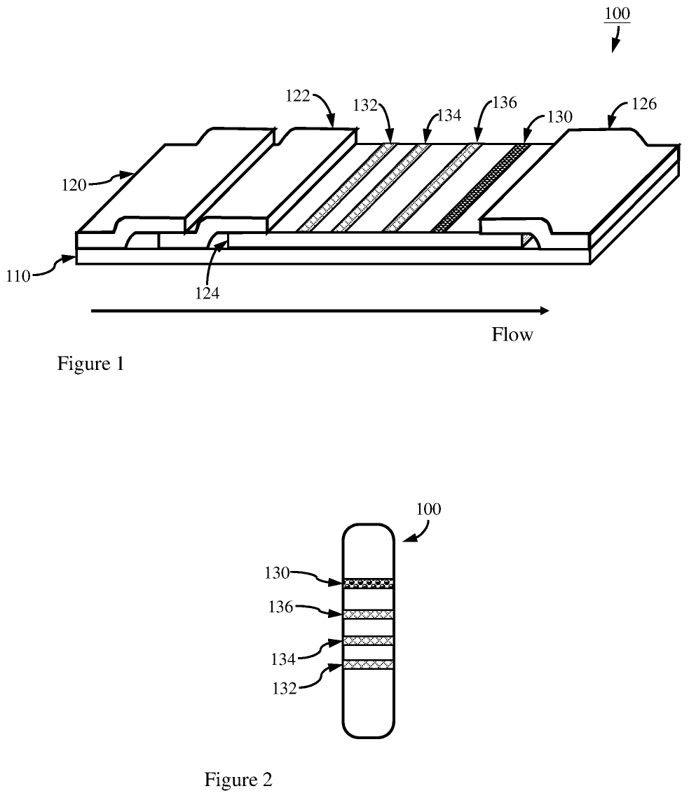

The sample pad 120, as shown in FIG. 1, acts as a sponge and holds an excess of sample fluid. Once soaked, the fluid flows to the second conjugate pad 122 in which the manufacturer has stored freeze dried bio-active particles called conjugates in a salt-sugar matrix. The conjugate pad 122 contains all the reagents required for an optimized chemical reaction between the target molecule (e.g., an antigen) and its chemical partner (e.g., antibody) that has been immobilized on the particle's surface. This marks target particles as they pass through the pad and continue across to the test lines 132, 134, and 136 and control line 130 on a nitrocellulose membrane 124. For example, in the home pregnancy test, the test line 132 shows a signal, often a color as in pregnancy tests. The control line 136 contains affinity ligands which show whether the sample has flowed through and the bio-molecules in the conjugate pad are active. After passing these reaction zones, the fluid enters the final porous material, the absorption pad 126 or wick, that simply acts as a waste container. All the sample pad 120, conjugate pad 122, nitrocellulose membrane 124, and absorption pad 126 are fastened to a soft adhesive board 110. FIG. 2 shows a top view of the rapid screening reagent 100.

In principle, any colored particle can be used, however latex (blue color) or nanometer-sized particles of gold (red color) are most commonly used. The gold particles are red in color due to localized surface plasmon resonance. Fluorescent or magnetic labelled particles can also be used, however these require the use of an electronic reader to assess the test result.

Sandwich assays are generally used for larger analytes because they tend to have multiple binding sites. As the sample migrates through the assay it first encounters a conjugate, which is an antibody specific to the target analyte labelled with a visual tag, usually colloidal gold. The antibodies bind to the target analyte within the sample and migrate together until they reach the test line. The test line also contains immobilized antibodies specific to the target analyte, which bind to the migrated analyte bound conjugate molecules. The test line then presents a visual change due to the concentrated visual tag, hence confirming the presence of the target molecules. The majority of sandwich assays also have a control line which will appear whether or not the target analyte is present to ensure proper function of the lateral flow pad.

Competitive assays are generally used for smaller analytes since smaller analytes have fewer binding sites. The sample first encounters antibodies to the target analyte labelled with a visual tag (colored particles). The test line contains the target analyte fixed to the surface. When the target analyte is absent from the sample, unbound antibody will bind to these fixed analyte molecules, meaning that a visual marker will show. Conversely, when the target analyte is present in the sample, it binds to the antibodies to prevent them binding to the fixed analyte in the test line, and thus no visual marker shows. This differs from sandwich assays in that no band means the analyte is present.

While not strictly necessary, most tests will incorporate a control line which contains an antibody that picks up free latex or gold in order to confirm the test has operated correctly. The control line is always as close to the absorption pad as possible.

Time to obtain the test result is a key driver for these products. Tests can take as little as a few minutes to develop. Generally there is a trade off between time and sensitivity:

more sensitive tests may take longer to develop. The other key advantage of this format of test compared to other immunoassays is the simplicity of the test, by typically requiring little or no sample or reagent preparation.

Lateral flow assays have a wide array of applications and can test a variety of samples like urine, blood, saliva, sweat, serum, and other fluids. They are currently used by clinical laboratories, hospitals, and physicians for quick and accurate tests for specific target molecules and gene expression. Other uses for lateral flow assays are food and environmental safety and veterinary medicine for chemicals such as diseases and toxins. LFTs are also commonly used for disease identification such as ebola, but the most common LFT is the home pregnancy test.

In the present invention, LFT can be applied to the RTI, such as COVID 19 and flu rapid screening test.

In a conventional art, samples can be obtained by various methods, including a nasopharyngeal swab, sputum (coughed up material), throat swabs, deep airway material collected via suction catheter or saliva. The likelihood of detecting the virus depends on collection method and how much time has passed since infection.

Collecting saliva may be as effective as nasal and throat swabs, although this is not certain. Sampling saliva may reduce the risk for health care professionals by eliminating close physical interaction. It is also more comfortable for the patient. Quarantined people can collect their own samples. A saliva test's diagnostic value depends on sample site (deep throat, oral cavity, or salivary glands). Some studies have found that saliva yielded greater sensitivity and consistency when compared with swab samples.

An antigen is the part of a pathogen that elicits an immune response. Antigen tests look for antigen proteins from the viral surface. In the case of a coronavirus, these are usually proteins from the surface spikes. SARS-CoV-2 antigens can be detected before onset of COVID-19 symptoms (as soon as SARS-CoV-2 virus particles) with more rapid test results, but with less sensitivity than PCR tests for the virus.

Samples may be collected via nasopharyngeal swab, a swab of the anterior nares, or from saliva. The sample is then exposed to paper strips containing artificial antibodies designed to bind to coronavirus antigens. Antigens bind to the strips and give a visual readout. The process takes less than 30 minutes, can deliver results at point of care, and does not require expensive equipment or extensive training.

Swabs of respiratory viruses often lack enough antigen material to be detectable. This is especially true for asymptomatic patients who have little if any nasal discharge. Viral proteins are not amplified in an antigen test. According to the WHO the sensitivity of similar antigen tests for respiratory diseases like the flu ranges between 34% and 80%. “Based on this information, half or more of COVID-19 infected patients might be missed by such tests, depending on the group of patients tested,” the WHO said. While some scientists doubt whether an antigen test can be useful against COVID-19, others have argued that antigen tests are highly sensitive when viral load is high and people are contagious, making them suitable for public health screening. Routine antigen tests can quickly identify when asymptomatic people are contagious, while follow-up PCR can be used if confirmatory diagnosis is needed.

The most notable antibodies are IgM and IgG. IgM antibodies are generally detectable several days after initial infection, although levels over the course of infection and beyond are not well characterized. IgG antibodies generally become detectable 10-14 days after infection and normally peak around 28 days after infection. This pattern of antibody development seen with other infections, often does not apply to SARS-CoV-2, however, with IgM sometimes occurring after IgG, together with IgG or not occurring at all. Generally, however, median IgM detection occurs 5 days after symptom onset, whereas IgG is detected a median 14 days after symptom onset. IgG levels significantly decline after two or three months.

Lateral flow assays have played a critical role in COVID-19 testing as they have the benefit of delivering a result in 15-30 minutes. The systematic evaluation of lateral flow assays during the COVID-19 pandemic was initiated at Oxford University as part of a UK collaboration with Public Health England. A study which started in June 2020 in the United Kingdom, FALCON-C19, confirmed the sensitivity of some lateral flow devices (LFDs) in this setting. Four out of 64 LFDs tested had desirable performance characteristics; the Innova SARS-CoV-2 Antigen Rapid Qualitative Test, in particular, underwent extended clinical assessment in field studies and was found to have good viral antigen detection/sensitivity with excellent specificity, although kit failure rates and the impact of training were potential issues. After closure of schools in January 2021, biweekly LFTs were introduced in England for teachers, pupils, and households of pupils when schools re-opened on Mar. 8, 2021 for asymptomatic testing. Biweekly LFT were made universally available to everyone in England on Apr. 9, 2021. LFTs have been used for mass testing for COVID-19 globally and complement other public health measures for COVID-19.

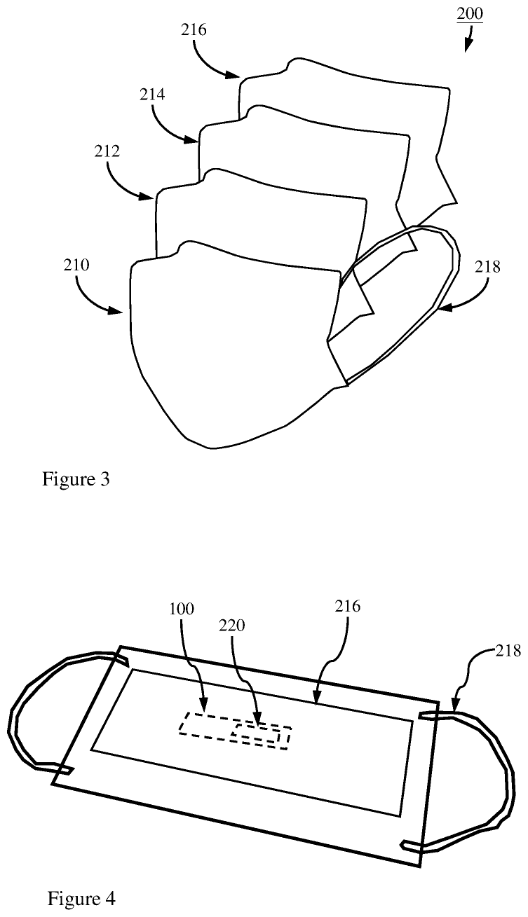

In the present invention, samples are collected in a mask or respirator. Please refer to FIG. 3, a mask or respirator 200 is illustrated. The mask 200 includes an outer layer 210, a filter layer 212, a support layer 214, and an inner layer 216 in sequence. The outer layer 210 is water repellent non-woven fabric, which can be polypropylene. The filter layer 212 is melt-blown non-woven fabric for filtering particles, and the support layer 214 is modacrylic for supporting the mask or respirator 200. In one embodiment, the filter layer 212 and the support layer 214 are optional or one of both is optional. Furthermore, sequence of the filter layer 212 and support layer 214 can be exchangeable. Some other layer can also be inserted between the outer layer 210 and the inner layer 216, such as an active carbon layer can also be inserted to absorb specific particles with peculiar smell. The inner layer 216 is skin-friendly non-woven fabric, which can be polypropylene. Two strings 218 are fastened to the outer layer 210 such that mask 200 can be hung on two ears.

Please refer to FIG. 4, a region 220 on the inner layer 216 is shown. This region 220 should be as close to the nostrils as possible and the rapid screening 100 is located inside the inner layer 216.

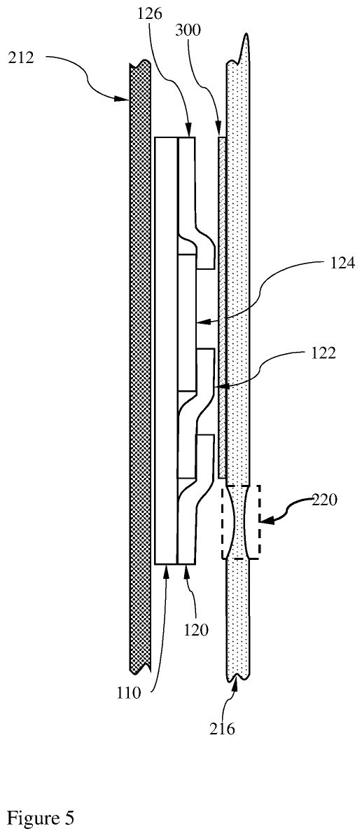

Please refer to FIG. 5, the sample pad 120 of the rapid screening reagent 100 is located to the region 220 to receive the air from the nostrils. A water-resist membrane 300, between the rapid screening reagent 100 and the inner layer 216, protect the conjugate pad 122, nitrocellulose membrane 124 and the absorption pad 126 from wetting, because the air from the nostrils contains a certain moisture. If the conjugate pad 122, nitrocellulose membrane 124 and the absorption pad 126 are wetting by the moisture from the nostrils, the test result may be prevail.

The rapid screening reagent 100 may be fastened to the inner layer 216 of the mask 200. The inner layer 216, in the present invention, should be somewhat transparent or semi-transparent, such that signals on the test lines can be shown directly and easily. The region 220 on the inner layer 216 should be a little thinner such that viruses in the air from the nostrils can pass through the region 220 more easily.

Because the air from the nostrils contains a certain moisture, the viruses in the air arriving the sample pad should include some moistures. Due the mask or respirator is worn several hours, the amounts of viruses and moistures accumulated in the sample pad 122 should trigger the lateral flow inside the rapid screening reagent 100. If the control line indicates no signal, a water or some fluids can be added to the sample pad to accelerate the lateral flow.

In this invention, the viruses in sample may cause the respiratory tract infection. Therefore, the test lines on the nitrocellulose membrane 124 can be one, two, three or more than three lines for detect several viruses at once. For example, conjugates on the conjugate pad 122 can include several reagents to bind with rhinovirus, coronavirus, influenza virus, respiratory syncytial virus, adenovirus, and/or parainfluenza. In another example, conjugates on the conjugate pad 122 can include several reagents to bind with HCoV-229E, HCoV-OC43, HCoV-NL63, HCoV-HKU1, SARS-CoV, MERS-CoV, and/or SARS-CoV-2. For example, a first reagent of conjugates may bind with SARS-CoV-2 and a first test line detects the first reagent, a second reagent of conjugates may bind with influenza virus and a second test line detects the second reagent, and a third reagent of conjugates may bind with HCoV family and a third test line detect the third reagent. In another example, variant reagents can bind corresponding variant protein of mutations of SARS-CoV-2.

In the present invention, the rapid screening reagent fastened on the mask can be wearable, such that any people can test themselves while wearing a mask or respirator.

In the present invention, the health-care or medical personnel working in nursing home or hospital may directly and easily test themselves if being infested by RTI viruses after taking off the mask or respirator. Especially for those personnel who has been infected asymptomatically.

In the present invention, swab is no longer necessary to collect samples in the nose, which may ease the uncomfortable way to collect samples.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

What is claimed is:

1. A respirator, comprising:
an outer layer and a transparent inner layer;
a screening reagent, between said outer layer and said inner layer, being fastened to said inner layer, wherein said inner layer includes a region for a sample pad of the screening reagent, such that gas from nostrils targets to the region; and
a water-resist membrane, between the screening reagent and said inner layer, covering the screening reagent except the region.

2. The respirator according to claim 1, further comprising a filter layer between said outer layer and said screening reagent, and a support layer between said filter layer and said screening reagent.

3. The respirator according to claim 2, wherein said outer layer is water repellent non-woven fabric, said filter layer is melt-blown non-woven fabric, said support layer is modacrylic, and said inner layer is skin-friendly non-woven fabric.

4. The respirator according to claim 2, wherein said outer layer and said inner layer are polypropylene.

5. The respirator according to claim 3, wherein said screening reagent comprises:
said sample pad for receiving the sample;
a conjugate pad including a first reagent for binding a first target molecule in the sample;
a nitrocellulose membrane including a first test line with a first antibody;
an absorption pad for making the sample flow through said conjugate pad and said nitrocellulose membrane by using capillary flow, thereby the first test line showing a first signal while the first reagent moving through the first test line; and
a soft adhesive board for fastening said sample pad, said conjugate pad, said nitrocellulose membrane, and said absorption pad.

6. The respirator according to claim 5, wherein the nitrocellulose membrane including a control line for testing the sample passing to the absorption pad.

7. The respirator according to claim 6, wherein the conjugate pad including a second reagent for binding a second target molecule in the sample, and the nitrocellulose membrane including a second test line with a second antibody.

8. The respirator according to claim 7, wherein the conjugate pad including a third reagent for binding a third target molecule in the sample, and the nitrocellulose membrane including a third test line with a third antibody.

9. The respirator according to claim 7, wherein the sample comprises a virus causing respiratory tract infection, and the virus is selected from the group consisting of rhinovirus, coronavirus, influenza virus, respiratory syncytial virus, adenovirus, and parainfluenza.

10. The respirator according to claim 9, wherein the coronavirus is selected from the group consisting of HCoV-229E, HCoV-OC43, HCoV-NL63, HCoV-HKU1, SARS-CoV, MERS-CoV, and SARS-CoV-2.

11. A wearable rapid screening reagent, comprising:
a mask including an outer layer and an inner layer, wherein the rapid screening reagent, between said outer layer and said inner layer, fastens to said inner layer, wherein said inner layer includes a region for a sample pad of the rapid screening reagent, such that gas from nostrils targets to the region; and
a water-resist membrane, between the rapid screening reagent and said inner layer, covering the rapid screening reagent except the region.

12. The wearable rapid screening reagent according to claim 11, wherein said mask comprises a filter layer between said outer layer and said screening reagent, and a support layer between said filter layer and said screening reagent.

13. The wearable rapid screening reagent according to claim 12, wherein said outer layer is water repellent non-woven fabric, said filter layer is melt-blown non-woven fabric, said support layer is modacrylic, and said inner layer is skin-friendly non-woven fabric.

14. The wearable rapid screening reagent according to claim 13, wherein said screening reagent comprises:
said sample pad for receiving the sample;
a conjugate pad including a first reagent for binding a first target molecule in the sample;
a nitrocellulose membrane including a first test line with a first antibody;
an absorption pad for making the sample flow through said conjugate pad and said nitrocellulose membrane by using capillary flow, thereby the first test line showing a first signal while the first reagent moving through the first test line; and
a soft adhesive board for fastening said sample pad, said conjugate pad, said nitrocellulose membrane, and said absorption pad.

15. The wearable rapid screening reagent according to claim 14, wherein the nitrocellulose membrane including a control line for testing the sample passing to the absorption pad.

16. The wearable rapid screening reagent according to claim 15, wherein the conjugate pad including a second reagent for binding a second target molecule in the sample, and the nitrocellulose membrane including a second test line with a second antibody.

17. The wearable rapid screening reagent according to claim 16, wherein the conjugate pad including a third reagent for binding a third target molecule in the sample, and the nitrocellulose membrane including a third test line with a third antibody.

18. The wearable rapid screening reagent r according to claim 17, wherein the sample comprises a virus causing respiratory tract infection, and the virus is selected from the group consisting of rhinovirus, coronavirus, influenza virus, respiratory syncytial virus, adenovirus, and parainfluenza.

19. The wearable rapid screening reagent according to claim 18, wherein the coronavirus is selected from the group consisting of HCoV-229E, HCoV-OC43, HCoV-NL63, HCoV-HKU1, SARS-CoV, MERS-CoV, and SARS-CoV-2.

20. A method for detecting virus, comprising:
providing a mask including an outer layer and an inner layer;
providing a screening reagent, between said outer layer and said inner layer, fastens to said inner layer, wherein said inner layer includes a region for a sample pad of the rapid screening reagent, such that gas from nostrils targets to the region;
providing a water-resist membrane, between the rapid screening reagent and said inner layer, covering the rapid screening reagent except the region;
wearing the mask;
checking if a signal of a test line on a nitrocellulose membrane of the screening reagent is shown.
